(19) United States
(12) Patent Application Publication
(10) Pub. No.: US 2012/0270849 A1
(43) Pub. Date: Oct. 25, 2012

(54) COMBINATIONS OF NIACIN, OMEGA-3 AND PLANT STEROLS/STANOLS FOR PREVENTION CHOLESTEROL TREATMENT

(75) Inventor: Dror Rom, Huntingdon Valley, PA (US)

(73) Assignee: ProSoft Software, Inc., Wayne, PA (US)

(21) Appl. No.: 13/452,111

(22) Filed: Apr. 20, 2012

Related U.S. Application Data
(60) Provisional application No. 61/477,407, filed on Apr. 20, 2011.
(60) Provisional application No. 61/564,538, filed on Nov. 29, 2011.

(51) Int. Cl. A61K 31/575; A61P 7/00

(52) U.S. Cl. 514/171

(57) ABSTRACT

Provided herein are nutritional compositions, regimens and methods for the prevention, mitigation, and treatment of a cholesterol abnormality. In some embodiments, the invention comprises combinations of niacin (precursor), and at least one ingredient selected from the group consisting of: an Omega-3 fatty acid, Omega-3 fatty acid precursor, or Omega-3 fatty acid derivative, and: a plant sterol, a plant sterol precursor, or a plant sterol derivative. In other embodiments, the combination includes all three of those ingredients.

### CROSS REFERENCE TO RELATED APPLICATIONS

This application claims the benefit of U.S. Provisional Application No. 61/477,407, filed Apr. 20, 2011 and U.S. Provisional Application No. 61/564,538, filed Nov. 29, 2011, both of which are incorporated by reference in their entireties as if fully set forth.

### BACKGROUND OF THE INVENTION

Cholesterol is a waxy, fat-like substance made in the liver and other cells and found in certain foods, such as food from animals, like dairy products, eggs, and meat. The body needs a limited amount of cholesterol in order to function properly (cell walls need cholesterol to produce hormones, vitamin D, and the bile acids that help to digest fat). However, when too much cholesterol is present in the body health problems such as heart disease may develop.

Excess cholesterol may lead to plaque (a thick, hard deposit) forming in the body's arteries narrowing the space for blood to flow to the heart. Over time, this buildup may cause atherosclerosis (hardening of the arteries) which can lead to heart disease. A heart attack may occur when not enough oxygen-carrying blood reaches the heart if a portion of the heart is completely cut off by a total blockage of a coronary artery.

Cholesterol travels through the blood attached to a protein. The combination of the protein and the cholesterol is known as a lipoprotein. Lipoproteins are classified as high density, low density, or very low density, depending on how much protein there is in relation to fat. Very low density lipoproteins (VLDL) are similar to LDL cholesterol in that it contains mostly fat and not much protein. High density lipoproteins (HDL) also referred to as “good” cholesterol, helps the body get rid of bad cholesterol in the blood. The vast body of scientific evidence support the following: 1. The higher the level of HDL cholesterol, the better. If your levels of HDL are low, your risk of heart disease increases. 2. Low density lipoproteins (LDL) also referred to as “bad” cholesterol, can cause buildup of plaque on the walls of arteries. The more LDL there is in the blood, the greater the risk of heart disease.

Triglycerides are another type of fat that is carried in the blood by very low density lipoproteins. Excess calories, alcohol, or sugar in the body are converted into triglycerides and stored in fat cells throughout the body. Your total cholesterol is a combination of your HDL, LDL and triglycerides, such that total cholesterol=HDL+LDL+(0.2×Triglycerides).

There are two common types of cholesterol tests that may be performed in order to determine if you have unhealthy cholesterol levels. A non-fasting cholesterol test will show your total cholesterol and HDL cholesterol. A fasting cholesterol test, called a lipid profile or a lipoprotein analysis, will measure your LDL, HDL, total cholesterol, and triglycerides. A non-fasting test may be performed first to determine if there is a potential problem (e.g., high total cholesterol, low HDL, high total cholesterol/HDL ratio) and if there is a potential problem then the non-fasting test may be performed.

Prevailing cholesterol guidelines define a total cholesterol number of less than 200 to be good. The guidelines also consider an HDL of greater than 40 for men and greater than 50 for women to be good. An LDL of less than 100 is ideal, while values between 130 and 159 are considered borderline and over 160 are considered high. Triglycerides of less than 150 are considered good. An LDL/HDL ratio of less than 3 is considered good and a total cholesterol/HDL ratio of less than 5 is considered good. The generally accepted cholesterol guidelines are summarized in FIG. 1.

If your cholesterol falls outside the cholesterol guidelines, and changing diet and/or exercise is not sufficient to bring your cholesterol within the guidelines, medications and/or dietary supplements may be utilized to help manage cholesterol levels. Several types of prescription drugs are often used to treat cholesterol; they include for example the class of drugs known as “statins”. While these drugs have been shown to be quite effective in the treatment of (primarily) LDL cholesterol, they are also known or suspected of causing severe side effects. Other medications and dietary supplements are sometimes used to treat cholesterol, among them are: Niacin (prescription or non-prescription) to increase HDL; dietary supplementation with Plant Sterols/Stanols to lower LDL; and Omega 3 (prescription or non-prescription) to lower Triglycerides. These medications and dietary supplements are considered safer by many practitioners since their side effects are generally milder compared to other classes of cholesterol-lowering prescription drugs (e.g. statins). However, when these medications and dietary supplements are taken individually, their effects may be negligible or small, and insufficient when the cholesterol level is markedly out of range.

For all these reasons, there exists a continuing and unmet need for safe, effective compositions and methods for controlling cholesterol levels in mammals that avoids or mitigate deleterious side effects, maintain known desirable effects, and provide novel and unexpected benefits in mammals.

### BRIEF DESCRIPTION OF THE DRAWINGS

The features and advantages of the various embodiments will become apparent from the following detailed description in which:

FIG. 1 is a table summarizing generally accepted cholesterol guidelines.

FIG. 2 is a table summarizing dosage ranges for various cholesterol medications.

FIG. 3 illustrates example in range and out of range cholesterol measurements for implementing a regimen, according to one embodiment.

FIG. 4 illustrates an example flowchart for determining an appropriate baseline regimen to be provided to a patient, according to one embodiment.

FIG. 5 illustrates the drug regimen that may be applied based on the cholesterol measurements being in or out of range, according to one embodiment.

FIG. 6 illustrates an example flowchart for adjusting a baseline (or current) regimen to be provided to a patient, according to one embodiment.

### SUMMARY OF THE INVENTION

Provided herein are nutritional compositions, regimens and methods for the control of cholesterol in mammals,.

In one embodiment, provided is a composition for the prevention, mitigation, or treatment of cholesterol abnormalities, the composition comprising of niacin or a niacin precursor or a niacin derivative, and at least one ingredient selected from the group consisting of: an Omega-3 fatty acid, Omega-3 fatty acid precursor, or Omega-3 fatty acid derivative, and: a plant sterol, a plant sterol precursor, or a plant sterol derivative.

In another embodiment, provided is a regimen for the prevention, mitigation, or treatment of cholesterol abnormalities, the regimen comprising the step of administering to a mammal niacin or a niacin precursor or a niacin derivative, and at least one ingredient selected from the group consisting of: an Omega-3 fatty acid, Omega-3 fatty acid precursor, or Omega-3 fatty acid derivative, and: a plant sterol, a plant sterol precursor, or a plant sterol derivative.

In yet another embodiment, provided is a method of preventing, mitigating, or treating a cholesterol abnormality in a mammal, the regimen comprising the step of administering to the male mammal an Omega-3 fatty acid, Omega-3 fatty acid precursor, or Omega-3 fatty acid derivative, and: a plant sterol, a plant sterol precursor, or a plant sterol derivative.

In yet another embodiment, provided is a regimen consisting of niacin precursor, or a niacin derivative and at least one of two components: an Omega 3, an Omega-3 fatty acid, Omega-3 fatty acid precursor, or Omega-3 fatty acid derivative, and: a plant sterol, a plant sterol precursor, or a plant sterol derivative. which when taken according to the method below, will prevent, mitigate, or treat a cholesterol abnormality.

In still another embodiment, provided is a method of applying a regimen to prevent, mitigate, or treat a cholesterol abnormality in a mammal. In this example, the method includes steps of: administering niacin or a niacin precursor or a niacin derivative and at least one ingredient selected from the group consisting of: measuring a subject's cholesterol; determining which cholesterol factors (HDL, LDL, triglycerides) are out of a selected range; administering a regimen comprising a selected dosage of niacin or a niacin precursor or a niacin derivative and at least one ingredient selected from the group consisting of: an Omega-3 fatty acid, Omega-3 fatty acid precursor, or Omega-3 fatty acid derivative, and: a plant sterol, a plant sterol precursor, or a plant sterol derivative; again measuring the subject's cholesterol; and repeating the steps of administering and measuring until the subject's cholesterol is within a preselected range. In an example of this embodiment, the selection of either Omega 3 or plant sterols is associated with bringing an associated cholesterol factor within range for each cholesterol factor out of range; applying the regimen to the patient; monitoring the patients cholesterol at defined periods of time (e.g., every 3 months); determining which cholesterol factors are out of range after application of the regimen; and adjusting the regimen to increase the dosage of the components associated with each cholesterol factor still out of range, wherein no change is made to components where the associated cholesterol factor is now within range, and wherein if a cholesterol factor is still out of range after a maximum dosage of the corresponding component has been applied seeking other options.

The methods can further comprise steps of diagnosing any male subject with a cholesterol abnormality, and administering the regimen until the abnormality is prevented, mitigated, or treated thereby.

### DETAILED DESCRIPTION OF THE PREFERRED EMBODIMENTS

The ensuing detailed description provides preferred exemplary embodiments only, and is not intended to limit the scope, applicability, or configuration of the invention. Rather, the ensuing detailed description of the preferred exemplary embodiments will provide those skilled in the art with an enabling description for implementing the preferred exemplary embodiments of the invention. It being understood that various changes may be made in the function and arrangement of elements without departing from the spirit and scope of the invention, as set forth in the appended claims.

To aid in describing the invention, directional terms are used in the specification and claims to describe portions of the present invention (e.g., upper, lower, left, right, etc.). These directional definitions are merely intended to assist in describing and claiming the invention and are not intended to limit the invention in any way. In addition, reference numerals may be introduced in a specification in association with a drawing figure and these may be repeated in one or more subsequent figures without additional description in the specification in order to provide context for other features.

As used herein, “niacin” (also referred to generally and herein as vitamin B₃, nicotinic acid and vitamin PP) is an organic compound having the IUPAC name pyridine-3-carboxylic acid and the formula C₆H₅NO₂ and, depending on the definition used, one of the forty to eighty essential human nutrients. It is a colorless, water-soluble solid is a derivative of pyridine, with a carboxyl group (COOH) at the 3-position. Other forms of vitamin B₃ include the corresponding amide, nicotinamide (“niacinamide”), where the carboxyl group has been replaced by a carboxamide group (CONH₂), as well as more complex amides and a variety of esters. The terms “niacin”, “nicotinamide”, and “vitamin B₃” are used interchangeably to refer to any member of this family of compounds, since they have similar biochemical activity. “Niacin” as used herein therefore further includes precursors of niacin such as nicotinamide, since both compounds can be converted to NAD and NADP in vivo. It also includes derivatives of niacin, such as the dietary supplement known as inositol hexanicotinate (IHN), which is inositol that has been esterified with niacin on all six of inositol's alcohol groups. IHN is usually sold as “flush-free” or “no-flush” niacin in units of 250, 500, or 1000 mg/tablets or capsules. It is sold as an over-the-counter formulation, and often is marketed and labeled as niacin. This form of niacin may not cause as much flushing as is associated with the immediate-release products.

As used herein, “plant sterol” and “plant stanol” means any sterol or stanol naturally occurring in plants, or derived from a naturally occurring plant material. This expressly includes stanol esters, a heterogeneous group of chemical compounds derived from starting materials such as phytosterols from plants. For example, phytosterols can be first hydrogenated to give a plant stanol, which is then esterified with a mixture of fatty acids, such as fatty acids derived from plants. Plant stanol esters are also found naturally occurring in small quantities in fruits, vegetables, nuts, seeds, cereals, legumes, and vegetable oils. Stanol ester is often added to rapeseed oil based margarine or other foods for its health benefits. Studies have indicated that consumption of about 2 grams per day provides a reduction in LDL cholesterol of about 10%. The compound itself passes through the gut without entering the blood stream or lymph. Its presence, however, reduces both the amount of cholesterol the body absorbs from food and the reabsorption of the cholesterol component of bile. By way of non-limiting example, a stanol ester is marketed by the Raisio Group under the trade name Benecol. Sterol esters compounds have the same effect as stanol esters on LDL, but they are partially absorbed by the body. The effects of higher serum plant sterol levels are so far not completely understood.

As used herein, “Omega-3” means Omega-3 fatty acids (also popularly referred to as ω-3 fatty acids or n-3 fatty acids), including those found naturally occurring in marine and plant oils. They are polyunsaturated fatty acids with a double bond (C═C) starting after the third carbon atom from the end of the carbon chain. The fatty acids have two ends—the acid (COOH) end and the methyl (CH3) end. The location of the first double bond is counted from the methyl end, which is also known as the omega (ω) end or the n end. Omega-3 fatty acids may have health benefits and are considered essential fatty acids, meaning that they cannot be synthesized by the human body but are vital for normal metabolism. Though mammals cannot synthesize n-3 fatty acids, they have a limited ability to form the long-chain n-3 fatty acids including eicosapentaenoic acid (EPA, 20 carbons and 5 double bonds), docosahexaenoic acid (DHA, 22 carbons and 6 double bonds) and α-linolenic acid (ALA, 18 carbons and 3 double bonds). Common sources of Omega-3 fatty acids include fish oils, algal oil, squid oil and some plant oils such as echium oil and flaxseed oil.

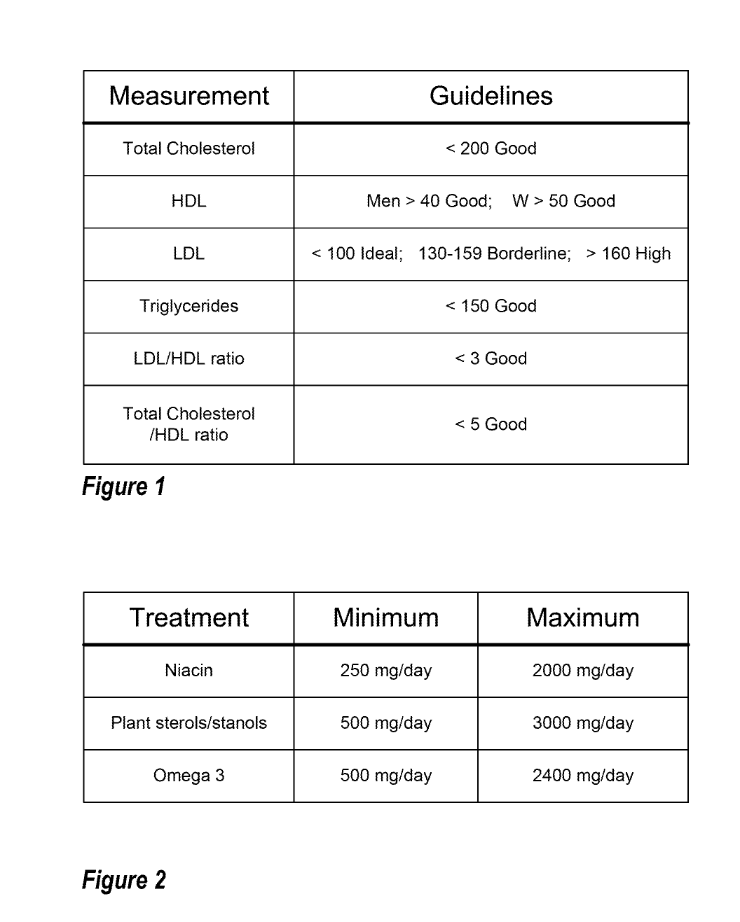

As used herein, “cholesterol abnormality” means any deviation in one or more medically recognized cholesterol factors or criteria, including but not limited to total cholesterol, triglycerides, HDL, LDL, and any ratios of any such cholesterol factors relative to one another. Further, a “cholesterol abnormality” as used herein may be temporary, transient, or permanent absent medical treatment or dietary treatment. Indeed, the methods herein are useful in adjusting any number or combination of cholesterol factors to accomplish the mitigation, treatment, or cure of a cholesterol abnormality for any desirable period of time, no matter whether for minutes, days, months or years. By way of non-limiting example, the Cholesterol Guidelines table of FIG. 1 illustrates some “normal” ranges for cholesterol and various cholesterol factors, as well as some currently medically recognized cholesterol abnormalities wherein one or more of cholesterol and/or cholesterol factors are outside of the normal range. For example, some abnormalities include, but are not limited to: total cholesterol of over 200; HDL under 40 for men and under 50 for women; LDL over 130 and in another example over 160 as “high” LDL; triglycerides over 150; an dLDL to HDL ratio of over 3 to 1; and a ratio of total cholesterol to HDL of over 5. The use of “low” “high” and “normal” herein as it relates to cholesterol and cholesterol factors is intended as relative to then-accepted medically recognized measurements of each factor, for particular mammals of particular gender, as well as having particular physiological, anatomical, and pharmacological profiles based upon such things as disease state, age, prescription and diet, among other things.

Provided herein are nutritional compositions, regimens and methods for the prevention, mitigation, and treatment of a cholesterol abnormality. In all embodiments, the invention comprises a combination of any of the three ingredients of niacin, Omega-3 fatty acids, and plant sterols and/or plant stanols, including any precursors or derivatives of any of them.

In other embodiments, the invention comprises nutritional regimens wherein combinations of any of niacin, Omega-3 fatty acids, and plant sterols or plant stanols are administered to a human mammal at preselected times, and in preselected amounts so as to render a benefit to the subject that prevents, mitigates, and/or treats a cholesterol abnormality. In one example, the benefit rendered is at least one of lowered total cholesterol, altering of the ratio of HDL to LDL, or temporary adjustment of the ratio of HDL to LDL in general, or at particular times or during particular periods of human activity.

Several types of prescription drugs are often used to treat cholesterol; they include for example the class of drugs known as “statins”. While these drugs have been shown to be quite effective in the treatment of (primarily) LDL cholesterol, they are also known or suspected of causing severe side effects, including causing or worsening some other symptoms or physiological conditions. Other medications and dietary supplements are sometimes used to treat cholesterol, among them are: niacin (prescription or non-prescription) to increase HDL; dietary supplementation with plant sterols/stanols to lower LDL; and dietary supplementation with Omega 3 to lower triglycerides. These medications and dietary supplements are considered safer as their side effects are generally milder compared to other prescription drugs used for lowering cholesterol. For patients with low unhealthy levels of HDL, niacin may positively affect cholesterol by increasing HDL and consequently decreasing both LDL/HDL and Total Cholesterol/HDL ratios. For patients with high unhealthy levels of LDL, plant sterols and stanols may positively affect cholesterol by lowering LDL and consequently decreasing LDL/HDL and total cholesterol/HDL ratios. For patients with high unhealthy levels of triglycerides, Omega 3 may positively affect cholesterol by lowering triglycerides and consequently decreasing total cholesterol/HDL ratio. Additionally, Omega 3 may also decrease VLDL and thus further improve cholesterol.

Blood vessel disease is often mediated by inflammatory processes. Evidence indicates that treatment with Omega 3 fatty acids, in combination with plant sterols/stanols, may have a synergistic anti-inflammatory effect, providing beneficial effect on cholesterol abnormalities and related conditions.

A regimen that lowers cholesterol may also have a long term beneficial effect on the prevention of narrowing of blood vessels. In each embodiment of the compositions, regimens and methods, the common feature is the presence of any two of the following three ingredients: niacin or a niacin precursor or derivative; an Omega-3 fatty acid or Omega-3 fatty acid precursor or derivative; and a plant sterol or plant stanol or a plant sterol or stanol precursor or derivative. Preferably, the compositions, regimens, and methods are characterized by the absence of any other drug indicated for treatment of a cholesterol abnormality. More preferably, the compositions, regimens and methods are characterized by the absence of any drug indicated for the treatment of cholesterol.

To the extent that control of cholesterol further aids in the prevention, mitigation, or treatment of cholesterol abnormalities, the following is relevant to the inventive aspects herein.

Depending on the tolerance and efficacy for an individual patient one or more of the above noted ingredients (niacin, Omega 3-fatty acids, plant sterols and/or plant stanols) or their precursors or derivatives may be utilized in an attempt to bring their cholesterol within the guidelines. FIG. 2 illustrates dosage ranges for these medications.

The dosage ranges shown in FIG. 2 are intended to be exemplary and are not intended to limit the scope of the invention. For example, the dosage range for niacin could be from 500 mg/day to 1500 mg/day instead of the range shown in FIG. 2. The dosage of any of the ingredients may be increased or decreased, depending on an individual patient's response and tolerance, again with the intent to maintain the dosage of each ingredient within its intended dose range as shown in FIG. 2.

The patient's blood cholesterol levels may be monitored at defined periods (e.g., every 3 months) and adjustments can be made to the regimen based on response to the baseline (or current) regimen. For example, a component may be added and/or a dose of a component may be changed (increased). If optimal levels for each of the cholesterol factors have been attained then no change is needed to the regimen. If sub-optimal levels are still present for one or more of the measurements then the doses for each associated supplement may be increased up to the maximum recommended dose. For example, the dose of niacin may be increased from 500 mg/day to 2000 mg/day if HDLs are still too low. If optimal levels are not attained with maximum doses, other treatment options must be considered.

The dosage regimen discussed above may be implemented in various manners. For example, the dosage regimen may be implemented as instructions stored on a processor readable storage medium, where the instructions when executed by a processor cause the processor to implement the functions described in the flow charts above.

Depending on the cholesterol measurements for an individual patient one or more of the above noted medications (Niacin, Omega 3, Plant sterols and stanols) may be utilized in an attempt to bring their cholesterol within the guidelines. In order to determine if a particular medicine should be applied, a determination will be made as to whether the associated cholesterol factor is within range or not.

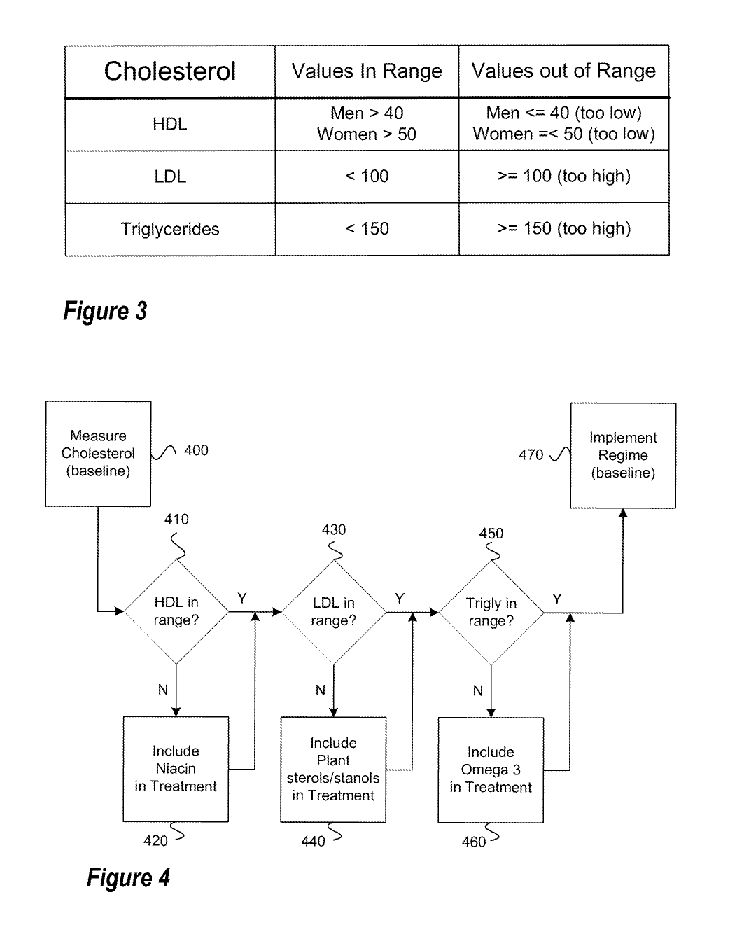

FIG. 3 illustrates in range and out of range cholesterol measurements that may be utilized for implementing a cholesterol medication regimen.

FIG. 4 illustrates an example flowchart that may assist in determining an appropriate regimen to be provided to a patient. Initially, the patients cholesterol is measured (baseline cholesterol) 400. A determination is then made as to whether the HDL measurement is within range 410. If the HDL measurement is not within range (e.g., an HDL value of 35 for either a male or female), then Niacin is included in their treatment regimen 420. Next a determination is then made as to whether the LDL measurement is within range 430. If the LDL measurement is not within range (e.g., an LDL value of 150), then Plant sterols/stanols are included in their treatment regimen 440. Next a determination is then made as to whether the Triglyceride measurement is within range 450. If the Triglyceride measurement is not within range (e.g., a Triglyceride value of 200), then Omega 3 is included in their treatment regimen 460. The overall treatment regimen (baseline treatment regimen) can then be implemented 470.

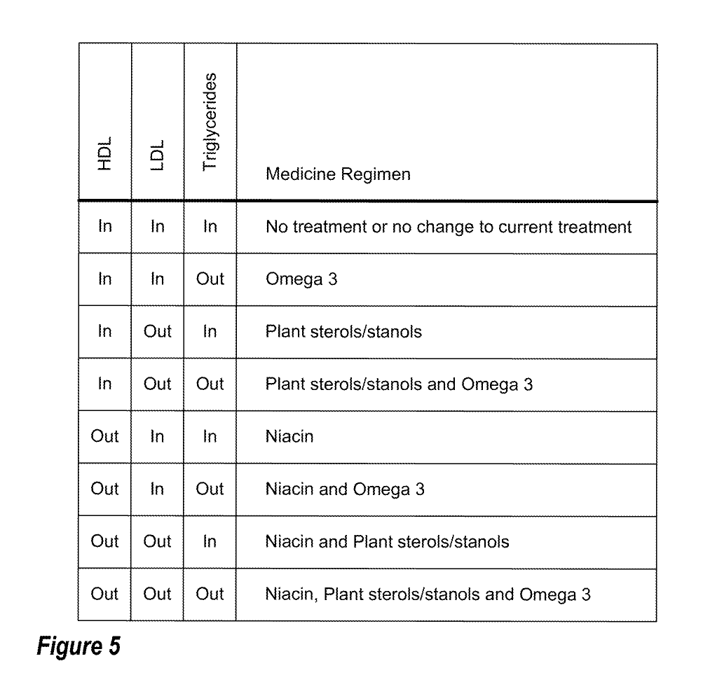

FIG. 5 illustrates the treatment regimen that may be applied based on the cholesterol factor being in or out of range. For example, if the baseline HDL, LDL and Triglycerides are all within range then no treatment regimen is required. However, if the baseline has all out of range the regimen may include Niacin, Plant sterols/stanols and Omega 3. The initial dosage for each of the medicines may start at the minimum values defined in FIG. 3.

The patient's blood cholesterol levels may be monitored at defined periods (e.g., every 3 months) and adjustments can be made to the regimen based on response to the baseline (or current) regimen. For example, a component may be added and/or a dose of a component may be changed (increased). If optimal levels for each of the cholesterol factors have been attained then no change is needed to the regimen. If sub-optimal levels are still present for one or more of the measurements then the doses for each associated supplement may be increased up to the maximum recommended dose. For example, the dose of niacin may be increased from 250 mg/day to 1000 mg/day, or alternatively from 500 mg/day up to as high as about 2000 mg/day, if HDLs are still too low. If optimal levels are not attained with maximum doses, other treatment options must be considered.

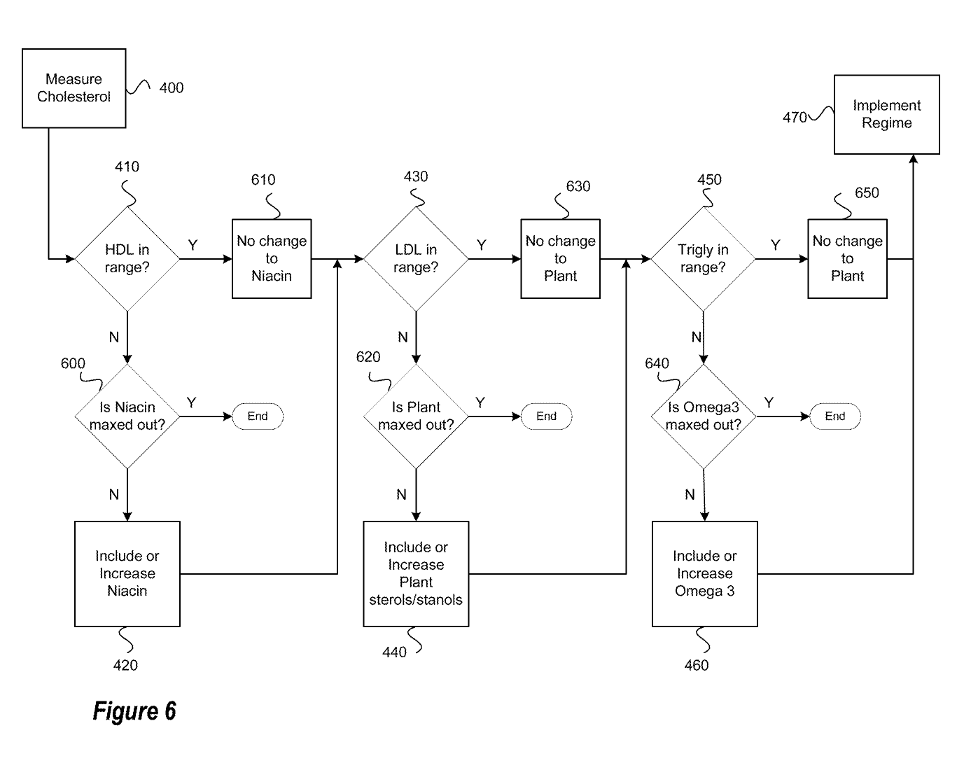

FIG. 6 illustrates an example flowchart for adjusting a baseline (or current) regimen to be provided to a patient. The flowchart is similar to the flowchart of FIG. 4 and the same reference numbers are used where the steps are the same. The additional steps include determining if the various components have reached a maximum dosage 600, 620, 640. If the dosage has not reached a maximum level, the dosage for that component can be increased or if the component was not part of the regimen it can be added thereto 420, 440, 460. If the dosage has reached a maximum level, then alternative treatments will be required. If the measurements are within range, then no changes are made to the dosage of the corresponding component 610, 630, 650.

The dosage regimen discussed above may be implemented in various manners. For example, the dosage regimen may be implemented as instructions stored on a processor readable storage medium, where the instructions when executed by a processor cause the processor to implement the functions described in the flow charts above.

The following is another example of a particular dosage regimen. The particular regimen depends on the measurement results concerning the subject's initial cholesterol (preferably including all cholesterol factors).

Niacin—starting dose of 250 mg/day, titrated up to 2000 mg/day

Omega 3—if baseline triglycerides>=150, 500 to 2400 mg/day,

Plant sterols/plant stanols—if baseline LDL>=100, 500 to 3000 mg/day.

Applicant has recently commenced clinical studies to further explore the inventive concepts herein. Among other things, Applicant has designed a human clinical trial protocol for fuller evaluation of the effect of administering the regimens and compositions described herein to humans having high cholesterol.

Further advantages of the regimens and methods herein include less side effects that may result from the substitution of the regimen over current cholesterol medications such as statins, and/or by the use of a lower dose of such current drugs in combination with the regimens and methods herein.

Still further advantages include a shorter time to effect on cholesterol conditions and related symptoms based upon the surprising synergy of the niacin, Omega-3 fatty acids, and plant sterols/stanols in controlling cholesterol, and in mitigating or treating cholesterol factors that may or may not be associated with high cholesterol, which is enhanced since the methods provide a customized regimen based on individual patient's initial cholesterol diagnosis and symptoms.

Further, it is expected that the novel regimens involving combinations of plant sterols, stanols, Omega-3 fatty acids will reduce the side effects of niacin, including flushing associated with administration of niacin alone.

While the principles of the invention have been described above in connection with preferred embodiments, it is to be clearly understood that this description is made only by way of example and not as a limitation of the scope of the invention.

1. A composition for the prevention, mitigation, or treatment of a cholesterol abnormality, the composition comprising:
a first ingredient selected from the group of: niacin, a niacin precursor, and a niacin derivative;
a second ingredient selected from the group of: an Omega-3 fatty acid, an Omega-3 fatty acid precursor and an Omega-3 fatty acid derivative; and
a third ingredient selected from the group of: a plant sterol, a plant sterol precursor, and a plant sterol derivative.

2. The composition of claim 1, wherein the first ingredient is inositol hexanicotinate.

3. A regimen for the prevention, mitigation, or treatment of a cholesterol abnormality in a mammal, the regimen comprising the steps of:
administering to the mammal a first ingredient selected from the group of: niacin, a niacin precursor, and a niacin derivative, a second ingredient selected from the group of: an Omega-3 fatty acid, an Omega-3 fatty acid precursor and an Omega-3 fatty acid derivative and a third ingredient selected from the group of: a plant sterol, a plant sterol precursor, and a plant sterol derivative.

4. The regimen of claim 3, wherein the cholesterol abnormality comprises at least one abnormality consisting of high LDL, low HDL, and high triglycerides, and wherein the step of administering niacin is performed until HDL reaches a predetermined HDL range.

5. The regimen of claim 4, wherein the first ingredient is administered as inositol hexanicotinate.

6. The regimen of claim 4, wherein the first ingredient is niacin is provided as a flush-free formulation.

7. The regimen of claim 4, further comprising administering the third ingredient until LDL measured in the mammal reaches a predetermined LDL range.

8. The regimen of claim 4, wherein the step of administering the second ingredient until the high triglycerides reach a predetermined triglycerides range.

9. The method of claim 4, wherein the step of administering at least one additional ingredient includes administering at least one plant sterol or plant stanol until the high LDL reaches a predetermined LDL range, and wherein the step of administering at least one additional ingredient further comprises administering at least one of an Omega 3, Omega-3 precursor, or Omega-derivative until the high triglycerides reach a predetermined triglycerides range.

10. The method of claim 9, wherein the regimen does not require concurrent administration of any statin or other cholesterol modifying pharmaceutical.

11. The method of claim 9, wherein the regimen optionally further includes concurrent administration of a statin or other cholesterol modifying pharmaceutical.

12. A method of preventing, mitigating, or treating a cholesterol abnormality in a mammal, the method comprising the steps of:
a. measuring a patient's cholesterol and determining any cholesterol factors are out of a preselected range;
b. administering a regimen comprising niacin and at least one ingredient selected from either:
i. the group consisting of: Omega-3 fatty acids, Omega-3 fatty acid precursors, or Omega-3 fatty acid derivatives, or
ii. the group consisting of: plant sterols, plant sterol precursors, and plant sterol derivatives,
c. wherein the regimen is associated with bringing an associated cholesterol factor within range for each cholesterol factor out of range.

13. The method of claim 12, further comprising the steps of:
a. monitoring the patients' cholesterol at preselected times;
b. at each preselected time, determining which cholesterol factors are out of a desired range; and adjusting the regimen to modify each cholesterol factor still out of the desired range, whereby the condition of a cholesterol abnormality is prevented, mitigated, or treated thereby.

14. The method of claim 13, further comprising:
a. measuring a patient's cholesterol;
b. determining which cholesterol factors (HDL, LDL, Triglycerides) are out of range;
c. adding to a regimen a dosage of a component selected from the groupo consisting of nNiacin, plant sterols, plant stanols, and Omega 3 fatty acis to bring a selected associated cholesterol factor within range for each cholesterol factor out of range;
d. applying the regimen to the patient;
e. monitoring the patients cholesterol at defined periods of time (e.g., every 3 months);
f. determining which cholesterol factors are out of range after application of the regimen; and
g. adjusting the regimen to increase the dosage of the components associated with each cholesterol factor still out of range, wherein no change is made to components where the associated cholesterol factor is now within range, and wherein if a cholesterol factor is still out of range after a maximum dosage of the corresponding component has been applied seeking other options.

15. The regimen of claim 14, wherein the step of adding to the regimen comprises administering at least one of an Omega 3, Omega-3 precursor, or Omega-derivative until the high triglycerides reach a predetermined triglycerides range.

16. The regimen of claim 14, wherein the step of adding to the regimen includes administering at least one plant sterol or plant stanol until the high LDL reaches a predetermined LDL range.

17. The regimen of claim 14, wherein the step of adding to the regimen includes administering at least one of an Omega 3, Omega-3 precursor, or Omega-derivative until the high triglycerides reach a predetermined triglycerides range.

18. The regimen of claim 14, wherein the regimen does not require concurrent administration of any statin or other cholesterol modifying pharmaceutical.

19. The regimen of claim 14, wherein the regimen optionally further includes concurrent administration of a statin or other cholesterol modifying pharmaceutical.

20. The regimen of claim 14, wherein the niacin is provided as at least one of inositol hexanicotinate or a flush-free niacin.
